(19) United States
(12) Patent Application Publication
(10) Pub. No.: US 2006/0178639 A1
(43) Pub. Date: Aug. 10, 2006

(54) Safety device for syringe needle

(76) Inventors: Fan Eric, Tanzi Hsiang (TW); Chung-Zong Huang, Taichung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/257,021

(22) Filed: Oct. 25, 2005

(30) Foreign Application Priority Data
Dec. 17, 2004 (CN) 200410101315.1

(51) Int. Cl. A61M 5/32

(52) U.S. Cl. 604/192

(57) ABSTRACT

A syringe device includes a barrel a needle set connected to one end of the barrel and the other end of the barrel is opened. A safety device has a sleeve and a first extension member which is movably mounted on the locking tip. The sleeve is slidably is mounted on the first extension member. The locking tip has a first flange the first extension member has a second flange extending therefrom respectively. The first extension member and the sleeve have a first recess and a second recess so that when the sleeve and the first extension member is pulled axially away from the barrel, the first and second flanges are respectively engaged with the first and second recesses to enclose the needle on the needle set.

### FIELD OF THE INVENTION

The present invention relates to a safety device for protecting the users from being injured by the needle of the syringe needle by axially pulling the extension member(s) of the safety device.

### BACKGROUND OF THE INVENTION

A conventional syringe device includes a barrel with a locking tip on one end thereof and a needle is connected to the locking tip. A plunger is movably inserted into the barrel from the other end of the barrel so that medicine can be pushed out from the needle. A cap can be mounted onto the locking tip to enclose the needle such that the needle is prevented from being contaminated. However, when re-capping the cap to enclose the needle after the syringe device is used, the users might be injured by the users. Besides, the janitors are possible victims by the injury of the needles when collecting the used syringe devices. Both of the situations are dangerous.

A safety device for syringe devices known to applicant is shown in FIGS. 16 and 17 and generally includes a sleeve 82 which is securely mounted to the locking tip 812 from which the needle 813 extends. The sleeve 82 includes a protrusion 82 extending outward from an outer periphery thereof. The syringe device 81 includes a barrel 811 with a plunger 814 movably inserted into the barrel 811 so as to push medicine in the barrel 811 out from the needle 813. A cap 831 can be mounted onto the sleeve 82 so as to enclose the needle 813 in the cap 831. The cap 831 includes an axial groove 832 and a transverse groove 835 which communicates with the axial groove 832. A locking space 837 is defined in one end of the transverse groove 835 with a separation rib so that the protrusion on the sleeve 82 can be locked within the locking space 837 to prevent the cap 831 from rotating. When using the syringe device, the user may rotating the cap 831 to force the protrusion on the sleeve 82 out from the locking space 837 and pulls the cap 831 toward the barrel 811 till the protrusion is in contact with an inner end 834 of the axial groove 832 and the needle 813 extends through a hole in a front end of the cap 831. After the syringe device is used, the user rotates the cap 831 in opposite direction and pulls the cap 831 away from the barrel 811 so that the needle 813 is received in the cap 831 again till the protrusion is in contact the end of the transverse groove 835, and the cap 831 is rotated again to let the protrusion to be located in either ends of the transverse groove 835. However, in order to facilitate the operation of the cap 831, the protrusion cannot be made too high which affects the rotation of the cap 831. Therefore, the cap 831 can be rotated unintentionally and the possibility of injury still exists.

The present invention intends to provide a safety device for protecting the users from injury by the needle, and the safety device is operated by way of pulling extension members which are locked at pre-determined positions.

### SUMMARY OF THE INVENTION

The present invention relates to a safety device for syringe device which comprises a barrel, a needle set connected to one end of the barrel and the other end of the barrel is opened. The needle set includes a locking tip and a needle extends from the locking tip. The safety device has a sleeve and a first extension member which is movably mounted on the locking tip. The sleeve is slidably mounted on the first extension member. The locking tip has a first flange and the first extension member has a second flange. The first extension member has a first recess and the sleeve has a second recess so as to receive the first and second flanges when the sleeve and the first extension member are pulled axially to enclose the needle in a space enclosed by the sleeve and the first extension member.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the first embodiment of the syringe device of the present invention;

FIG. 2 is a cross sectional view to show the safety device of the present invention;

FIG. 3 is a cross sectional view to show that the safety device of the present invention is pulled;

FIG. 4 is a cross sectional view, from another angle, of the safety device of the present invention is pulled;

FIG. 5 shows that a connection member is engaged with the barrel;

FIG. 6 shows a tube is inserted into the barrel;

FIG. 7 shows that liquid is collected via the collection member;

FIG. 8 shows the safety device is pulled to enclose the needle;

FIG. 9 is an exploded view to show the second embodiment of the syringe device of the present invention;

FIG. 10 shows that the cap is not yet removed from the needle set for the second embodiment of the syringe device of the present invention;

FIG. 11 shows that the safety device is pulled upward for he second embodiment of the syringe device of the present invention;

FIG. 12 shows a third embodiment of the syringe device of the present invention;

FIG. 13 is a cross sectional view to show the fourth embodiment of the syringe device of the present invention;

FIG. 14 shows that the safety device is pulled upward for the fourth embodiment of the syringe device of the present invention;

FIG. 15 is an exploded view to show the fourth embodiment of the syringe device of the present invention;

FIG. 16 is an exploded view to show a conventional syringe device and safety device, and

FIG. 17 shows a cross sectional view to the conventional safety device on the conventional syringe device.

### DETAILED DESCRIPTION OF THE PREFERRED EMBODIMENT

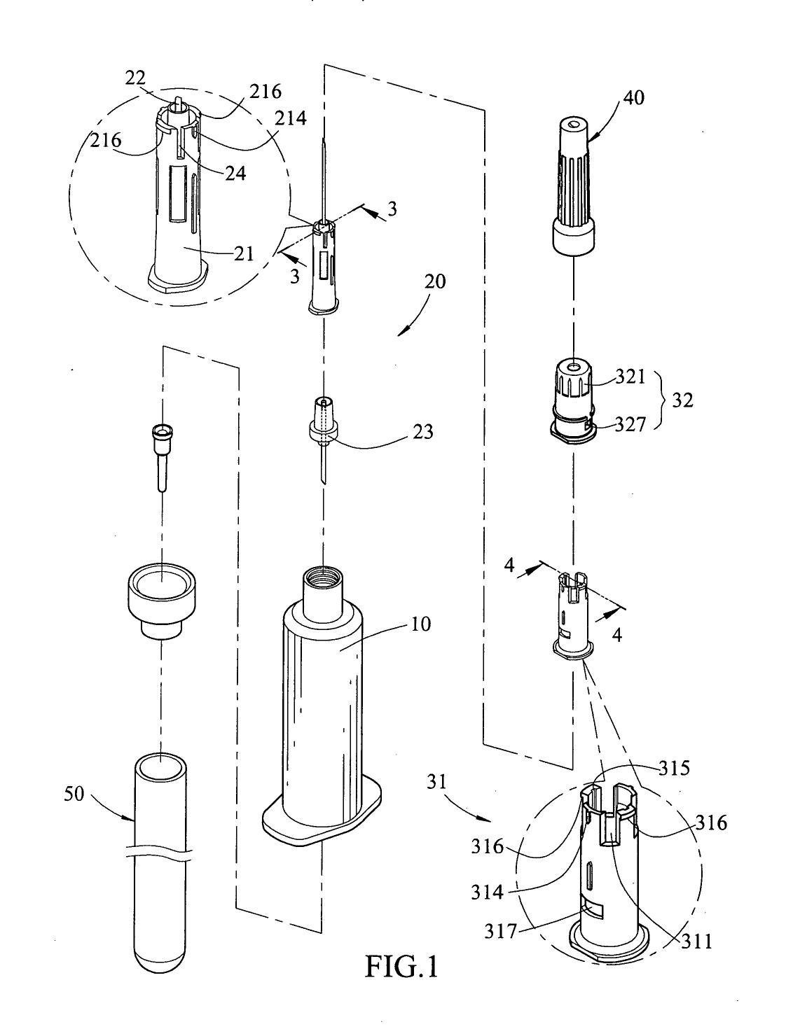

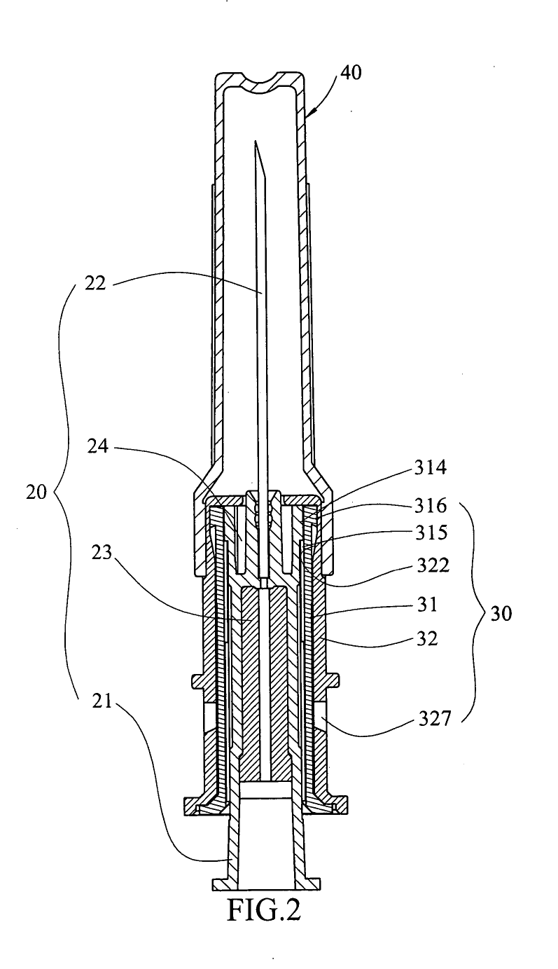

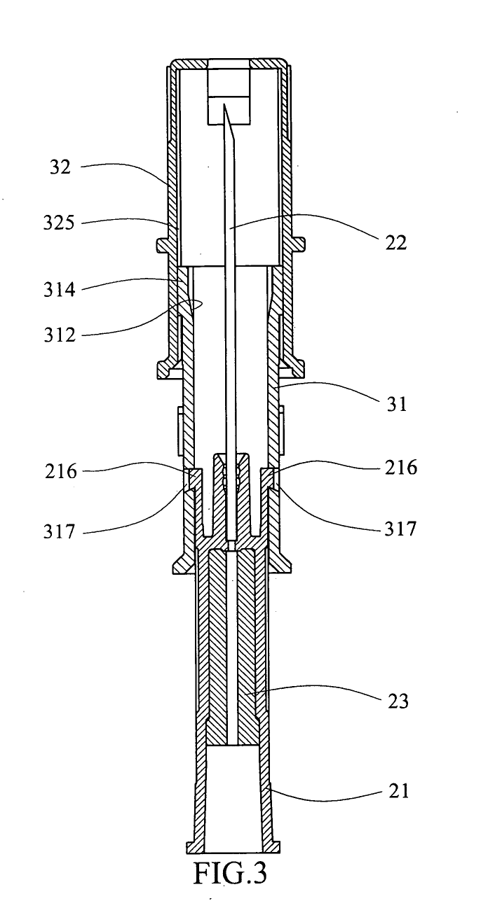

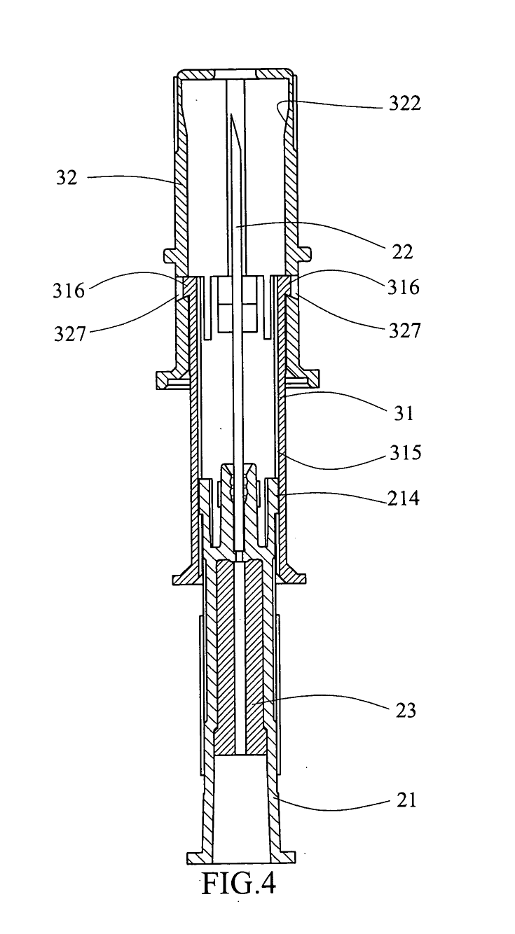

Referring to FIGS. 1 to 4, the syringe device of the present invention comprises a barrel 10 having a needle set 20 connected to one end of the barrel 10 and the needle set 20 includes a locking tip 21 and a needle 22 extends from the locking tip 21.

A safety device 30 includes a sleeve 32 and a first extension member 31 The first extension member 31 is movably mounted of the locking tip 21 and the sleeve 32 is slidably mounted on the first extension member 31. The sleeve 32 and the first extension member 31 can be pulled axially away from the barrel to enclose the needle 22. The locking tip 21 includes a first slot 24 which opens a top end of the locking tip 21 so that the top end of the locking tip 21 can be pushed inward.

The locking tip 21 has a first flange 216 and the first extension member 31 has a second flange 316 extending therefrom respectively. The first extension member 31 has a first recess 317 and the sleeve 32 has a second recess 327 so as to receive the first and second flanges 216, 316 when the sleeve 32 and the first extension member 31 are pulled. A cap 40 may be mounted to the sleeve 32 to enclose the needle 22 before the syringe device is used. The sleeve 32 has a first engaging portion 321 which are composed of a plurality of annular grooves and the cap 40 has a second engaging portion 41 which are composed of a plurality annular flanges which are engaged with the annular grooves.

The sleeve 32 includes a first inclined surface 312 defined in an inner periphery thereof and the first flange 216 on the lock tip 216 is located within a space between the inclined surface 312 and the sleeve 32. The first extension member 31 has a second slot 311 defined therein and opens to a top end thereof so that the top end of the first extension member 31 can be pushed inward. A second inclined surface 322 is defined in an inner periphery of the first extension member 31 so that the first flange 216 on the locking tip 21 is located between the second inclined surface and the locking tip 21 when the safety device is not yet pulled.

The locking tip 21 has a first positioning member 214 and the first extension member 31 has a second positioning member 314 on an outer periphery thereof. The first extension member 31 has a first guide groove 315 and the sleeve 32 has a second guide groove 325 so that the first and second positioning members 214, 314 are movably engaged with the first and second guide grooves 315, 325.

When the safety device is not pulled, the needle 22 is enclosed by the cap 40 and the locking tip 21, the first extension member 31 and the sleeve 32 are over lapped with each other and the first flange 216 and the second flange 316 are not squeezed. After use, the first extension member 31 and the sleeve 32 are pulled toward the needle 22, the first and second flanges 216, 316 are then squeezed inward by the first and second inclined surfaces 312, 322 till the first and second flanges 216, 316 are engaged with the first and second recesses 317, 327. The first and second flanges 216, 316 each have an inclined bottom surface and the first and second recesses 317, 327 each have an tilt surface so that the inclined bottom surfaces of the first and second flanges 216, 316 slip along the tilt surface of the first and second recesses 317, 327. By this way, the needle 22 is completely enclosed by the safety device while the user does not contact the needle 22 and the direction of the pulling of the safety device is from the barrel 10 toward the needle 22 so that the user does not hurt by the needle 22.

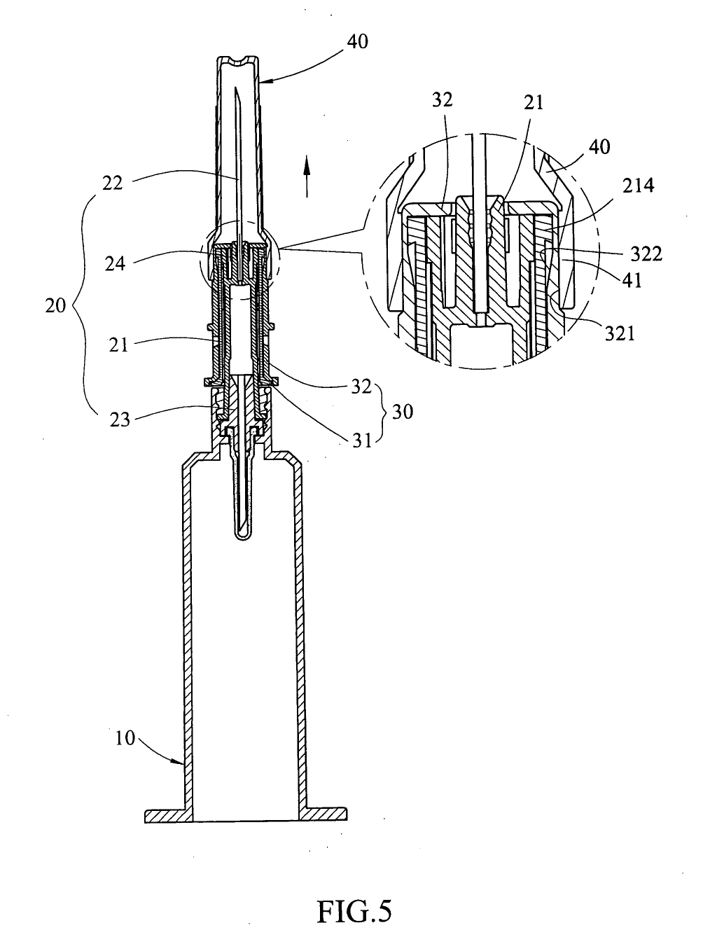

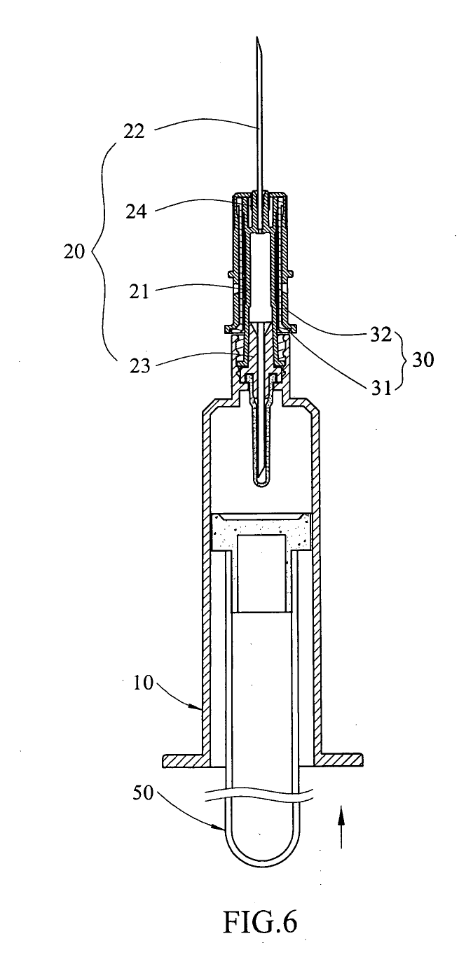

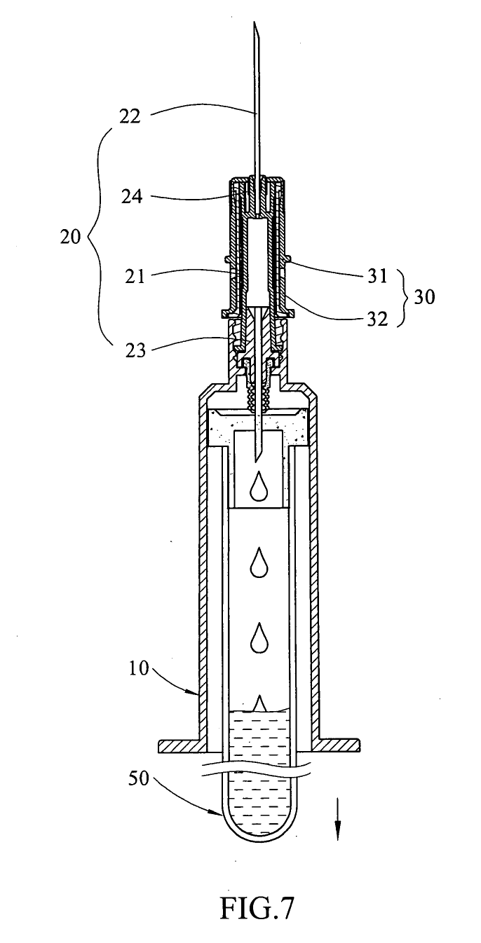

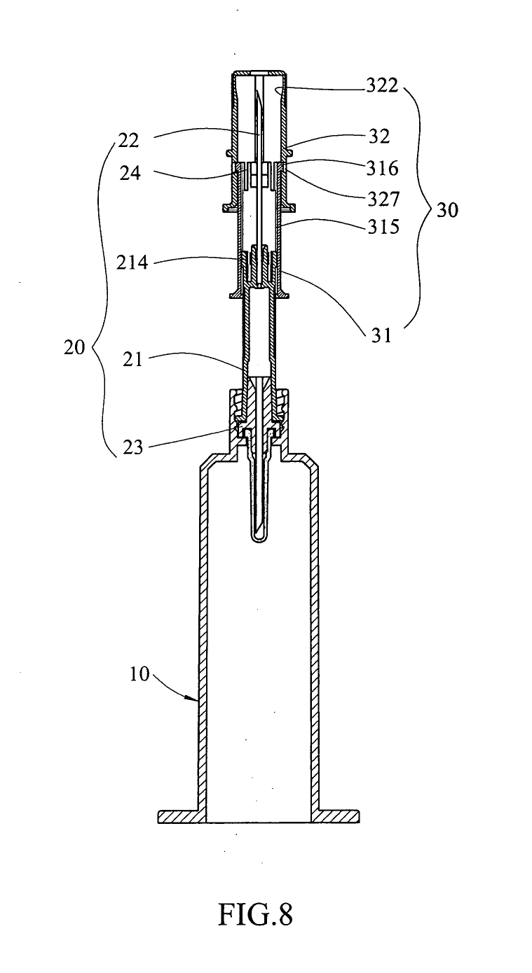

Referring to FIGS. 5 to 8, a connection member 23 can be engaged with the barrel 10, an outer periphery of the connection member 23 is securely engaged with an inner periphery of the locking tip 21. The connection member 23 may also be integrally connected to the inner periphery of the barrel 10. A tube 50 is inserted into the barrel 10 and the tube extending from the connection member 23 is inserted into the tube 50. The blood (for example) is then sucked from the needle 22 and collected into the tube 50 via the tube of the connection member 23. The tube 50 is then removed from the barrel 10. The safety device 30 is then pulled till the first and second flanges 216, 316 are engaged with the first and second recesses 317, 327 to enclose the needle 22.

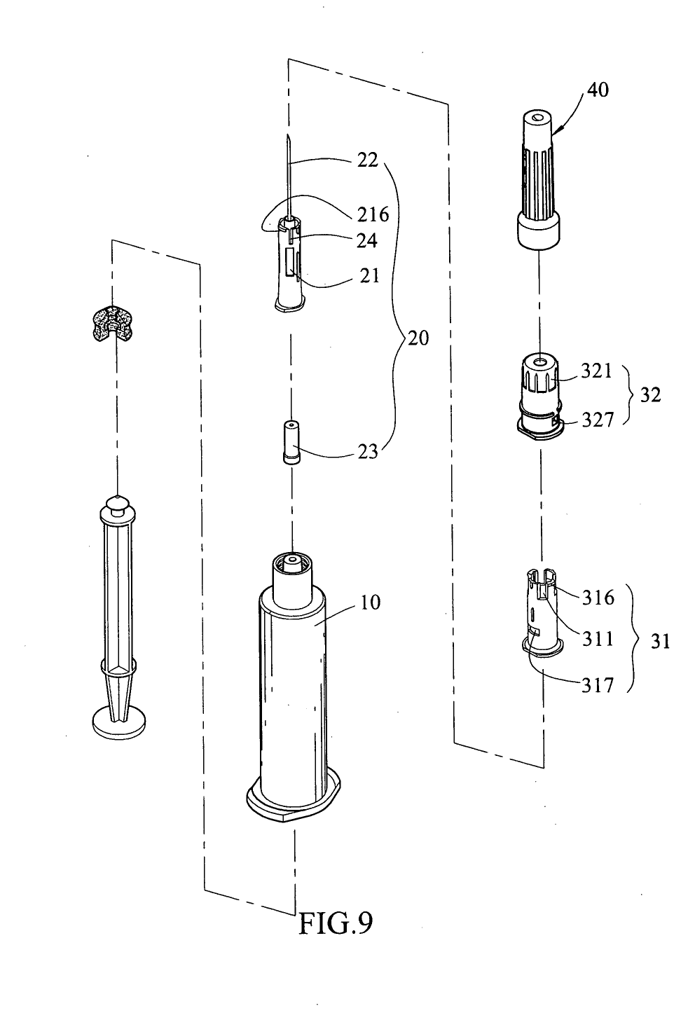

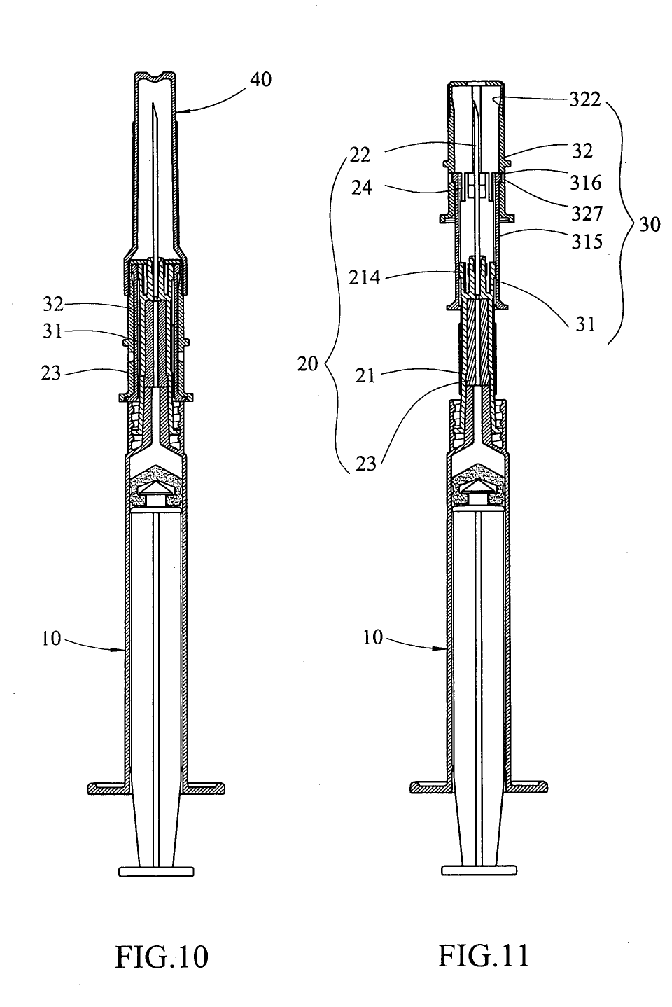

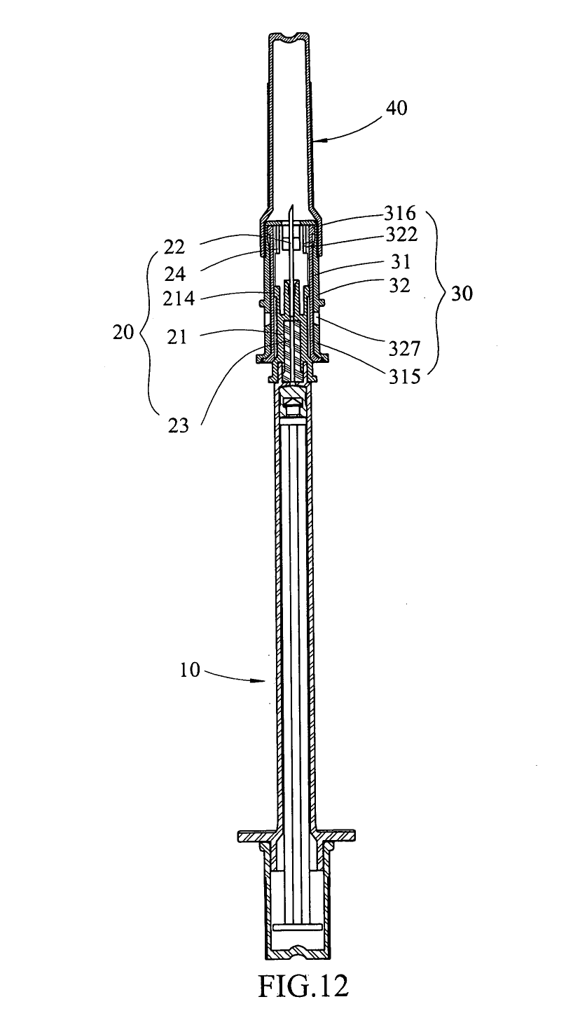

FIGS. 9-11 show a second embodiment of the present invention wherein a plunger is inserted into the barrel from the other end (opposite to the needle set) of the barrel 10. The medicine in the barrel 10 is pushed by pushing the plunger to eject from the needle 20. After use, the safety device 30 is operated to enclose the needle 22. FIG. 12 shows a third embodiment which shows that the syringe device can be used for insulin ejection.

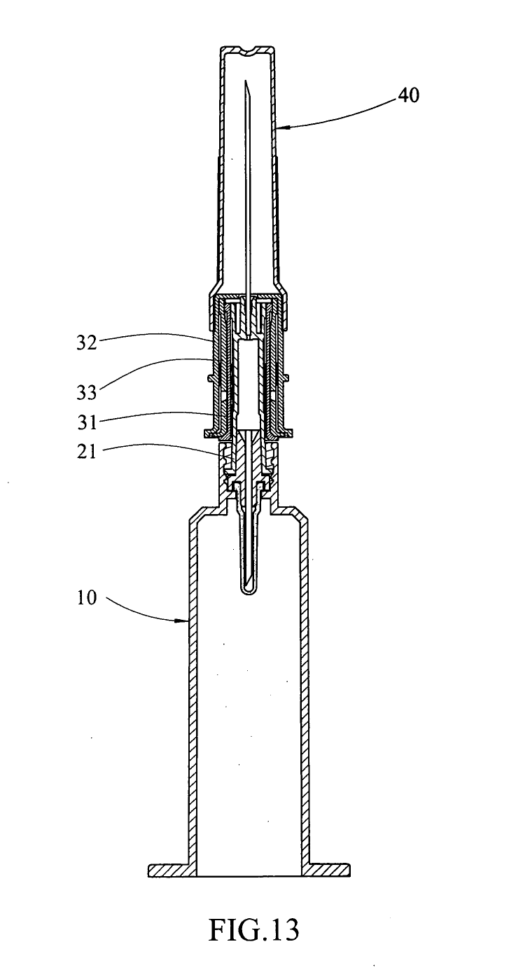

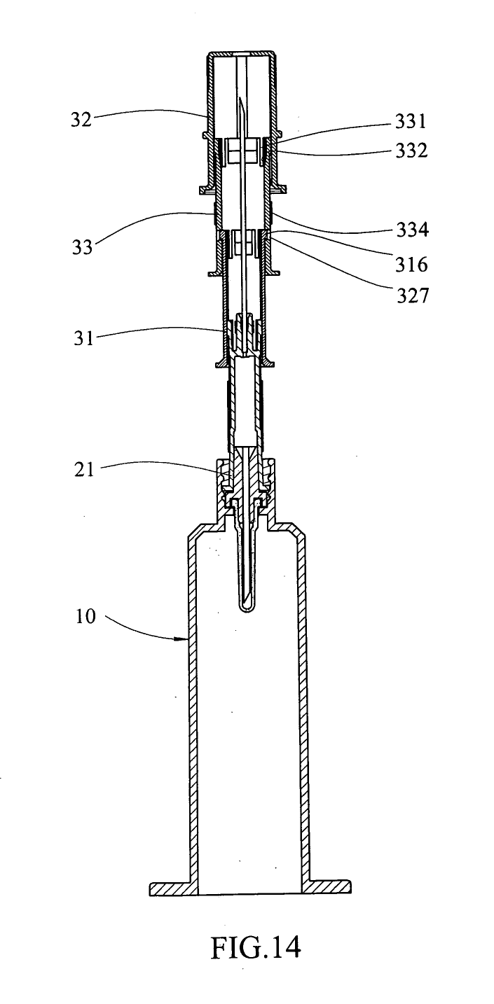

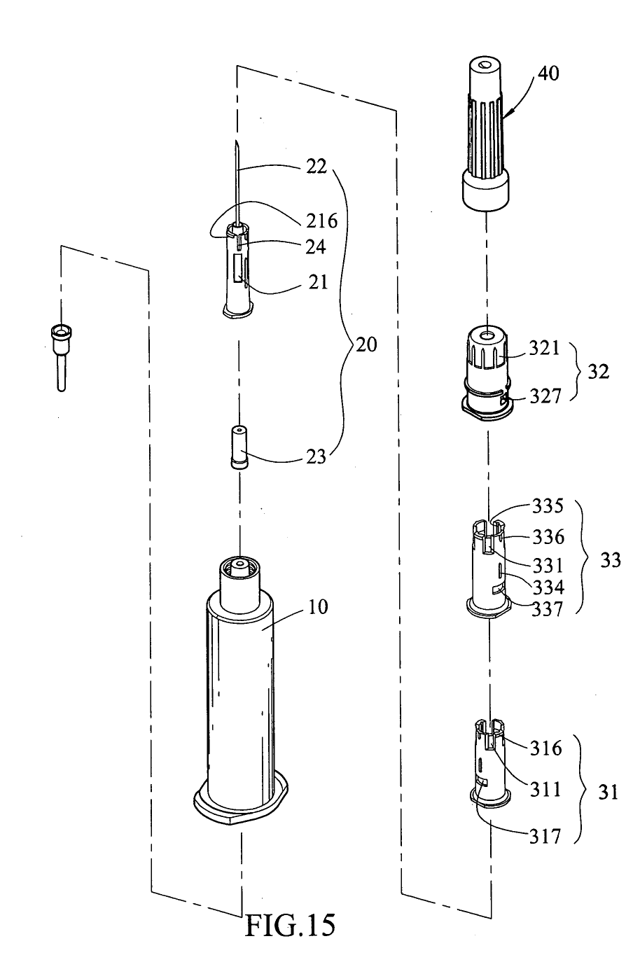

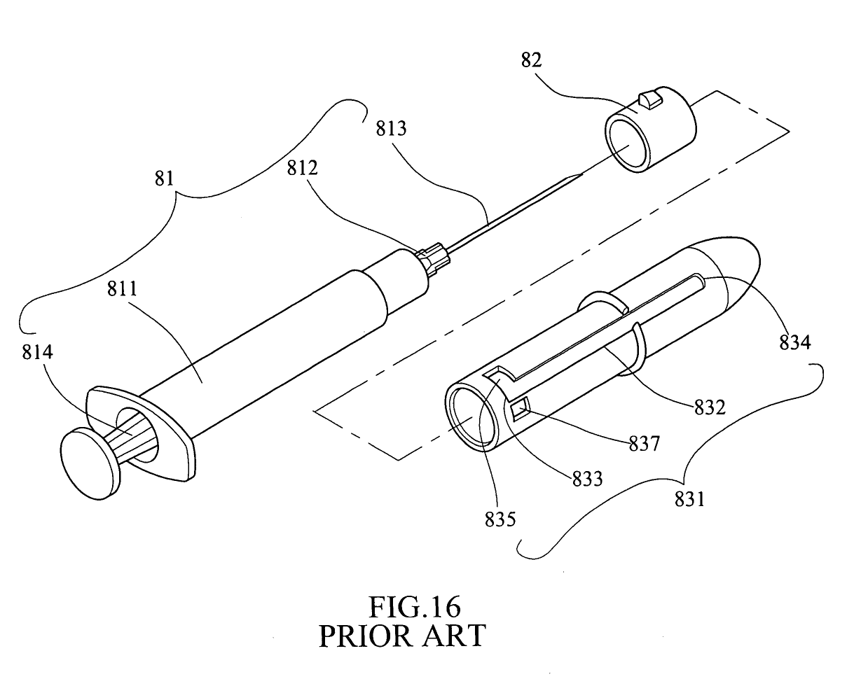

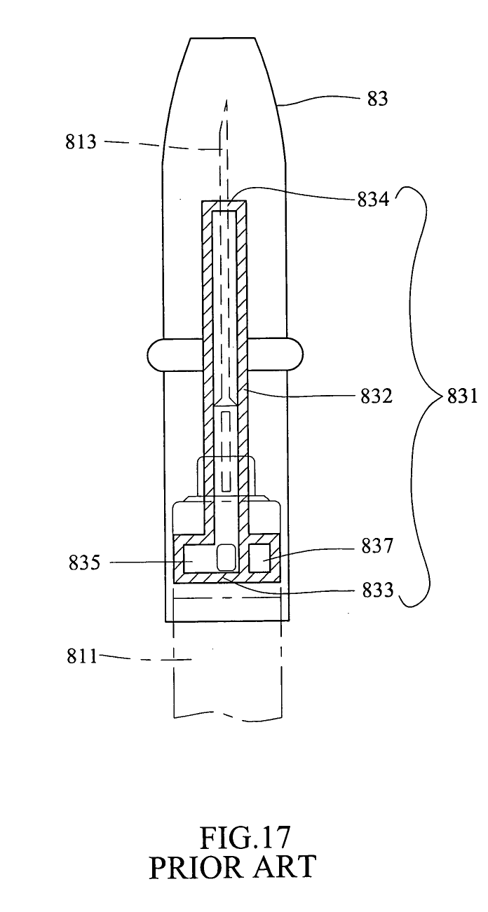

FIGS. 13 to 15 show a fourth embodiment of the present invention wherein the safety device 30 further includes a third extension member 33 which is located between the first extension member 31 and the sleeve 32. A third flange 336 extends from the third extension member 33 and a third recess 337 is defined in the third extension member 33. The second extension member 33 has a third positioning member 334 on an outer periphery thereof and the second extension member 33 has a third guide groove 335 so that the first, second and third positioning members 214, 314, 334 are movably engaged with the first, second and third guide grooves 315, 325, 335 when the safety device 30 is operated.

The second extension member 33 has a third slot 331 defined in a top thereof so that the when the second extension member 33 is mounted on the first extension member 31, the front end of the second extension member 33 is pushed inward. The second extension member 33 further includes a third inclined surface 332 defined in an inner periphery thereof so that the second flange 316 on the first extension member 31 is received between the third inclined surface 332 and the outer periphery of the first extension member 31 when the safety device is not yet operated.

The safety device 30 is short when the syringe device is not yet used so as to save storage space and the safety device 30 can be pulled to a sufficient length to enclose the needle 20 after use. When the safety device 30 is operated, the flanges 216, 316 are engaged with the recesses 317, 327 so that the user is acknowledged that the safety device 30 is completely extended. The top ends of the locking tip 21 and the safety device 30 can be quizzed inward so that the user can easily operate the safety device without too much effort.

While we have shown and described the embodiment in accordance with the present invention, it should be clear to those skilled in the art that further embodiments may be made without departing from the scope of the present invention.

What is claimed is:

1. A syringe device comprising:
a barrel having a needle set connected to one end of the barrel and the needle set including a locking tip and a needle extending from the locking tip;
a safety device having a sleeve mounted onto the locking tip, a flange extending from an outer periphery of a front end of the locking tip, a recess defined in the sleeve so that the flange is engaged with the recess, and
a cap mounted to the sleeve to receive the needle therein, the needle being received in the safety device when the cap is removed and the safety device is pulled axially away front the barrel.

2. The device as claimed in claim 1, wherein the sleeve includes a first inclined surface defined in an inner periphery thereof and the flange on the lock tip is located within a space between the inclined surface and the sleeve.

3. The device as claimed in claim 1, wherein the locking tip includes a positioning member on an outer periphery thereof and the sleeve includes a guiding groove with which the positioning member of the locking tip is slidably engaged.

4. A syringe device comprising:
a barrel having a needle set connected to one end of the barrel and the needle set including a locking tip and a needle extending from the locking tip;
a safety device having a sleeve and a first extension member which is movably mounted on the locking tip, the sleeve slidably mounted on the first extension member, the locking tip having a first flange and the first extension member having a second flange extending therefrom respectively, the first extension member having a first recess and the sleeve having a second recess so as to receive the first and second flanges, and
a cap mounted to the sleeve.

5. The device as claimed in claim 4, wherein the sleeve includes a first inclined surface defined in an inner periphery thereof and the first flange on the lock tip is located within a space between the inclined surface and the sleeve, the first extension member has a second slot defined therein and a second inclined surface defined in an inner periphery of the first extension member so that the first flange on the locking tip 21 located between the second inclined surface and the locking tip.

6. The device as claimed in claim 4, wherein the locking tip has a first positioning member and the first extension member has a second positioning member on an outer periphery thereof, the first extension member has a first guide groove and the sleeve has a second guide groove so that the first and second positioning members are movably engaged with the first and second guide grooves.

7. The device as claimed in claim 4, wherein the slot of the first extension member opens to a top end thereof so that the top end of the first extension member can be pushed inward.

8. The device as claimed in claim 4, wherein the safety device includes a third extension member which is located between the first extension member and the sleeve, a third flange extends from the third extension member and a third recess is defined in the third extension member.

9. The device as claimed in claim 8, wherein the locking tip has a first positioning member and the first extension member has a second positioning member on an outer periphery thereof, the second extension member has a third positioning member on an outer periphery thereof, the first extension member has a first guide groove, the sleeve has a second guide groove and the second extension member has a third guide groove so that the first, second and third positioning members are movably engaged with the first, second and third guide grooves.

10. The device as claimed in claim 8, wherein the second extension member has a third slot defined in a top thereof so that the when the second extension member is mounted on the first extension member, the front end of the second extension member is pushed inward.

11. The device as claimed in claim 8, wherein the second extension member includes a third inclined surface defined in an inner periphery thereof so that the second flange on the first extension member is received between the third inclined surface and the outer periphery of the first extension member.

12. The device as claimed in claim 4, wherein the locking tip includes a first slot which opens a top end of the locking tip so that the top end of the locking tip is pushed inward.

13. The device as claimed in claim 4, wherein the locking tip includes a connection member received therein and an outer periphery of the connection member is securely engaged with an inner periphery of the locking tip.

14. The device as claimed in claim 13, wherein the collection member is integrally connected with the locking tip.

15. The device as claimed in claim 4, wherein the sleeve has a first engaging portion which is composed of a plurality of annular grooves, the cap has a second engaging portion which are composed of a plurality annular flanges which are engaged with the annular grooves.

16. The device as claimed in claim 4, wherein the first and second flanges each have an inclined bottom surface and the first and second recesses each have an tilt surface so that the inclined bottom surfaces of the first and second flanges slip along the tilt surface of the first and second recesses.
